(19) United States
(12) Patent Application Publication
(10) Pub. No.: US 2026/0098294 A1
(43) Pub. Date: Apr. 9, 2026

(54) DETECTION OF MINIMAL RESIDUAL DISEASE AND CANCER RECURRENCE IN DRAIN FLUID

(71) Applicant: DROPLET BIOSCIENCES, INC., Cambridge, MA (US)

(72) Inventors: Wendy Winckler Adamovich, Melrose, MA (US); Seka Lazare, Melrose, MA (US)

(21) Appl. No.: 19/342,237

(22) Filed: Sep. 26, 2025

Related U.S. Application Data
(60) Provisional application No. 63/700,190, filed on Sep. 27, 2024.

(51) Int. Cl. C12Q 1/6869; C12Q 1/6806; C12Q 1/6809

(52) U.S. Cl. CPC C12Q 1/6869; C12Q 1/6806; C12Q 1/6809; C12Q 2600/106; C12Q 2600/118

(57) ABSTRACT

This disclosure relates to the detection of minimum residual disease (MRD) in surgical drain fluid. The data collected from the drain fluid is also useful in predicting the recurrence of cancer. Comparison is also made between the analysis results of plasma and drain fluid.

### BACKGROUND

Disease recurrence is a significant cause of mortality in cancer generally, and specifically in HPV-negative head and neck squamous cell carcinoma (HNSCC), with up to 50% of patients recurring within 2 years. No reliable methods exist to tailor adjuvant therapy for individual patients based on risk of recurrence after surgery. Moreover, there are no approved molecular tests to identify residual disease in the immediate post-surgical window when adjuvant therapy decisions should be made. While treatment selection considers pathological risk factors, such as HPV status, tumor stage or extranodal extension (ENE), 5-year overall survival rates remain low (10-61%, depending on HNSCC subtype). Treatment approaches, such as aggressive adjuvant radiotherapy (RT) or chemotherapy plus radiotherapy (CRT), have been associated with improvements in both progression free survival (PFS) and overall survival in HNSCC patients, but often result in high morbidity, as well as physiological, financial and emotional burden.

Thus, identifying those patients who would most benefit from escalated treatment remains a critical need. Detection of HPV viral DNA is prognostic in HPV-positive HNSCC. However, plasma cfDNA is not prognostic and has generally failed to detect recurrence. Moreover, ctDNA is generally first detected months after surgery and median first positive in HNSCC is found at about 33 days. Given the limitations of plasma-based MRD testing, there has been interest in examining more proximal biofluids, including urine, bronchioalveolar lavage fluid, CSF, and lymphatic drain fluid. The lymphatic system plays a critical role in the tumor microenvironment and is the primary route of metastatic spread, making it an ideal biospecimen to evaluate early MRD. Surgical drain fluid, which is primarily lymphatic fluid, is a good proximal source of biomarkers associated with cancer and is an overlooked source of diagnostic information. See, e.g., co-pending, co-owned application WO 2024/173105, incorporated by reference herein. There continues to exist a need in the art for improved methods to reliably detect MRD and recurrence via molecular testing.

### SUMMARY

The invention provides methods for predicting recurrence and minimal residual disease and for informing therapeutic choice and predicting therapeutic efficacy. The invention takes advantage of the insight that there exists diagnostic information from lymphatic exudate in proximity to a tumor that cannot be detected in significant amounts in a contemporaneously obtained blood sample. Accordingly, the invention allows early characterization and prognosis of disease that is critical in assessing patient status and a course of treatment.

In particular aspects, methods of the invention recognizes that cfDNA in lymphatic exudate is present in amounts between about 50-fold and 75-fold higher than can be found in blood. In particular, the invention provides that such cfDNA from lymphatic exudate is predictive of cancer recurrence and/or minimal residual disease earlier in time with respect to detection possible in blood. Thus, methods of the invention allow a timely decision on course of treatment and a more accurate view of disease progression at the earliest possible postsurgical stage.

In preferred methods, the invention comprises obtaining a lymphatic exudate in proximity to a tumor and analyzing cfDNA in the exudate that is substantially undetectable in blood. The lymphatic exudate cfDNA is sequenced and disease prognosis is predicted based on the sequence analysis of the cfDNA. Prediction of minimal residual disease, for example, is based on comparing cfDNA sequence obtained from lymphatic exudate to known sequences associated with disease outcome and/or recurrence. The known sequences may be obtained from a database of sequences, may be tumor-informed, or may be otherwise obtained empirically.

In one aspect, methods of the invention comprise using a tumor-informed SNV-based approach applicable to any solid tumor, analyzing MRD in lymphatic exudate from HPV-negative HNSCC patients postoperatively at about 24 hours. According to the invention, ctDNA in lymphatic exudate collected via surgical drains about 24 hours after surgery accurately identifies MRD and outperforms ctDNA analysis in plasma. According to the invention, proximal lymphatic exudate is enhanced in locoregional relapse early stage (I-II) patients. Moreover, matched analysis of plasma collected at this early post-surgical timepoint was not predictive of recurrence.

In general, the invention is based on the recognition that ctDNA indicative of minimal residual disease (MRD) in lymphatic exudate presents an early indication of MRD and recurrence that cannot be found in blood or plasma samples obtained within the same early timepoints at which the exudate is obtained. In general, the exudate is obtained about 48 hours or less post-surgery but may be obtained contemporaneous with a surgical procedure. The sensitivity to predicting MRD in methods of the invention is about 70% or greater for ctDNA found in the exudate and not found in plasma.

Methods of the invention are also applicable to non-nucleic acid markers present in lymphatic exudate and that are not present in plasma at similar timepoints. Examples of non-nucleic acid markers include proteins and peptides, carbohydrates, and metalloproteins.

According to methods of the invention, biomarkers identified in lymphatic exudate and that are not contemporaneously present in plasma are useful for disease diagnosis, prediction of MRD, recurrence, and for therapeutic selection and prediction of efficacy. In certain other aspects, methods of the invention are repeated over time in the lymphatic exudate and compared to plasma samples in order to assess disease progression and outcome. In addition, adjunct procedures, such as PET scans, CT scans, tissue pathology, MRI, X-ray and proteomic analysis are used in conjunction with ctDNA sequence analysis in lymphatic exudate to diagnose disease, predict recurrence, detect or predict MRD, inform therapeutic selection and/or predict therapeutic efficacy. In some aspects, the invention is used to assess cancer in HPV-negative hnscc.

Lymphatic exudate may be obtained through any convenient means, including an implanted drain or may be collected contemporaneous with a surgical procedure. The surgical procedure may be a dissection, a biopsy, an extraction or other invasive procedure that results in expulsion of lymphatic exudate.

Other aspects and advantages of the invention are apparent to the skilled artisan based on the following claims and detailed description thereof.

### BRIEF DESCRIPTION OF THE DRAWINGS

FIG. 1A shows a method for classifying a sample as MRD+ or MRD-based on its relationship to a calculated MRD cutoff.

FIGS. 1B-1E show that 24-hour post-operative lymph is enriched in ctDNA compared to plasma (n=42 patients).

FIG. 2 shows that lymph but not plasma ctDNA correlates with HNSCC recurrence.

FIGS. 3A-3E show that lymph ctDNA outperforms individual pathology features.

### DETAILED DESCRIPTION

The present disclosure provides methods for early identification of indicators of disease, presence or potential for MRD or recurrence, and therapeutic selection and/or efficacy. Methods of the invention take advantage of the insight that markers of disease are present in lymphatic exudate earlier than they would be present in blood or plasma. Thus, detection in lymphatic exudate provides an important diagnostic at a point that is critical for the assessment of disease progression.

Methods of the invention comprise obtaining lymphatic exudate and analyzing a biomarker that is present in the exudate and not in a contemporaneous plasma or blood sample. The biomarker may be any biomarker that is detectable in the exudate and not in a contemporaneously-obtained blood or plasma sample, including, but not limited to, nucleic acid (DNA, RNA) and proteins.

The invention provides an advance in the timing and accuracy of predicting disease status and course in a sample that provides diagnostic input that would not be available in another sample type (e.g., blood, urine, sputum, saliva, and the like).

### EXAMPLE 1

A cohort of 73 HPV-independent HNSCC patients were assessed as follows. Of the 73 patients, 36 patients were studied in an initial cohort and 37 were reserved for the replication cohort. There was no significant difference between initial and replication cohorts for available clinical data or demographic features. The median age of the study population was 63. 67% were male. 71.2% of patients received adjuvant therapy (1.4% CT, 46.6% RT, 20.5% CRT; 2.7% immunotherapy). The median follow up was 37.4 months. 34 out of 73 patients experienced disease recurrence (REC) (17 initial cohort and 17 replication cohort) and 39 out of 73 patients had no evidence of disease (NED) with at least one year of clinical follow up (19 initial and 20 replication cohort). 42 patients had matched plasma samples available (29 initial cohort, 13 replication cohort).

ctDNA in Lymph and Plasma Samples at 24 Hours Post-Surgery

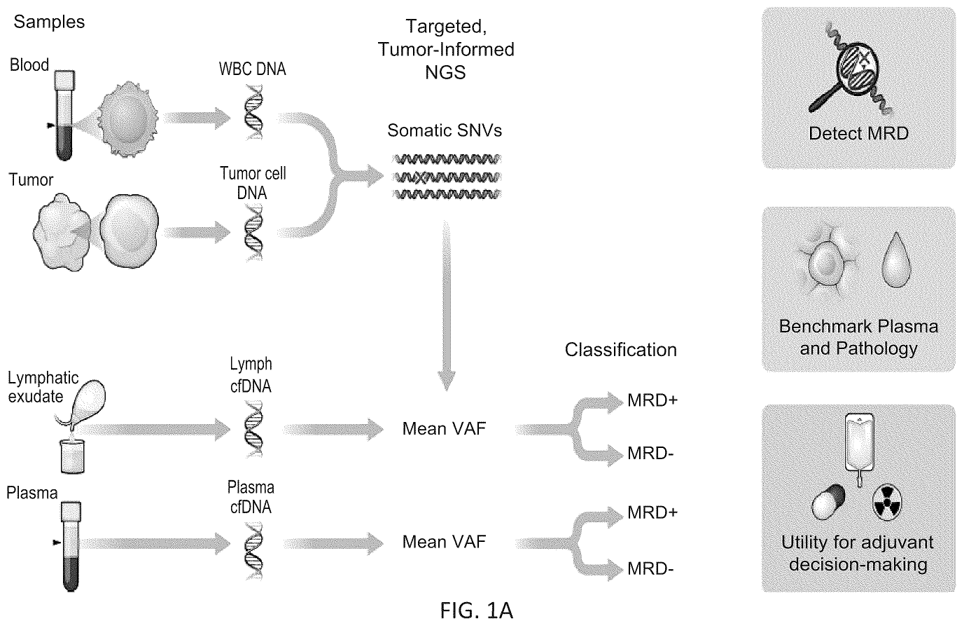

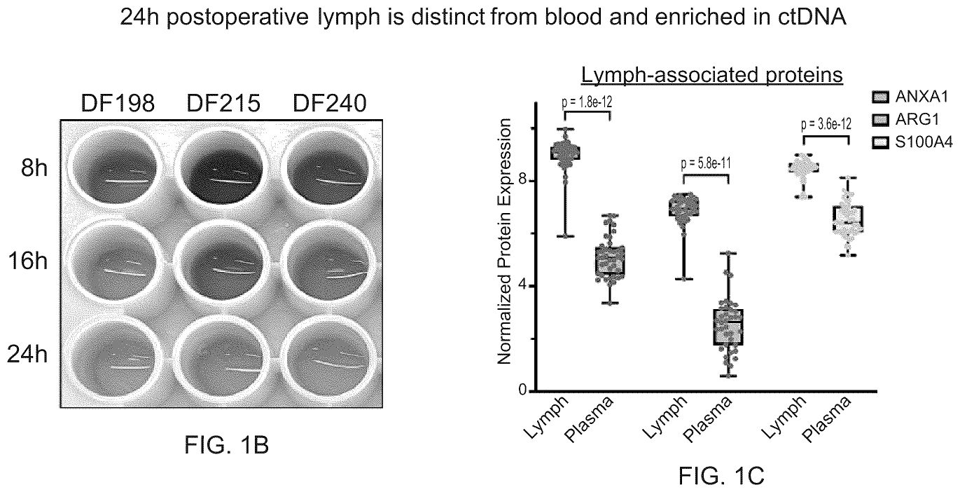

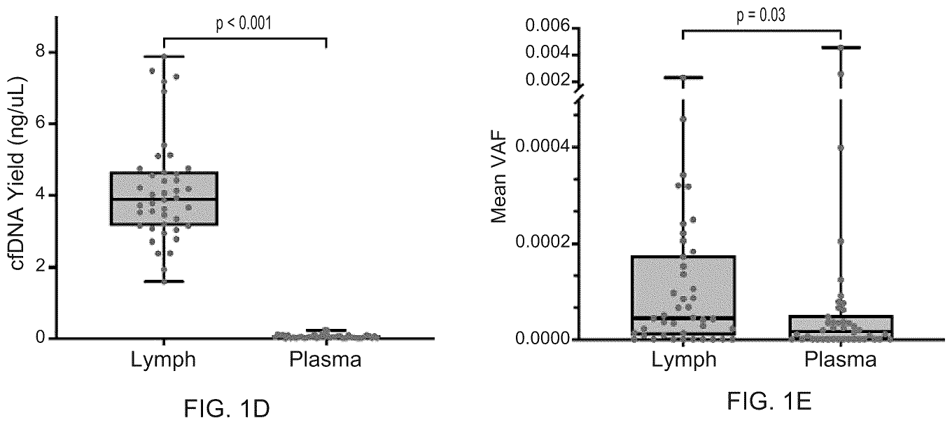

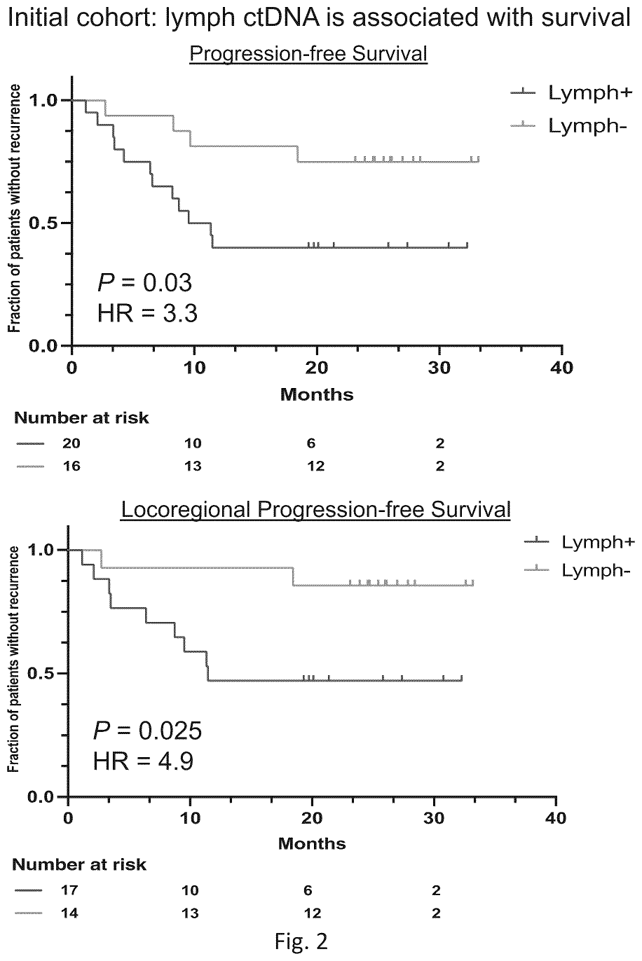

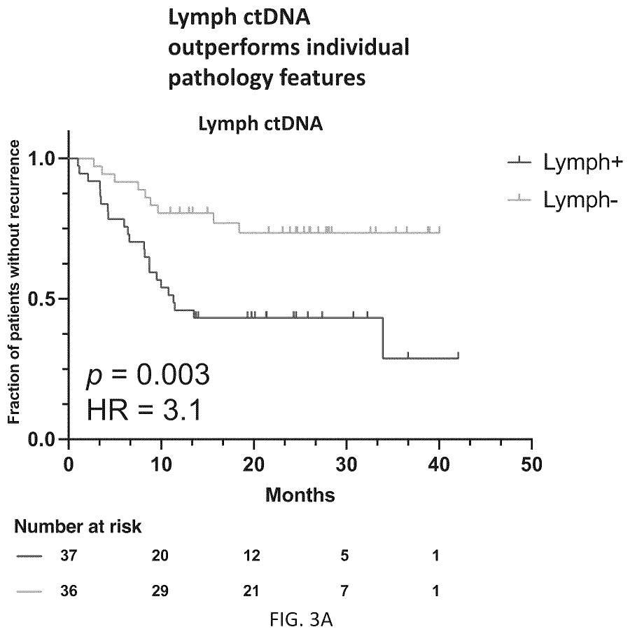

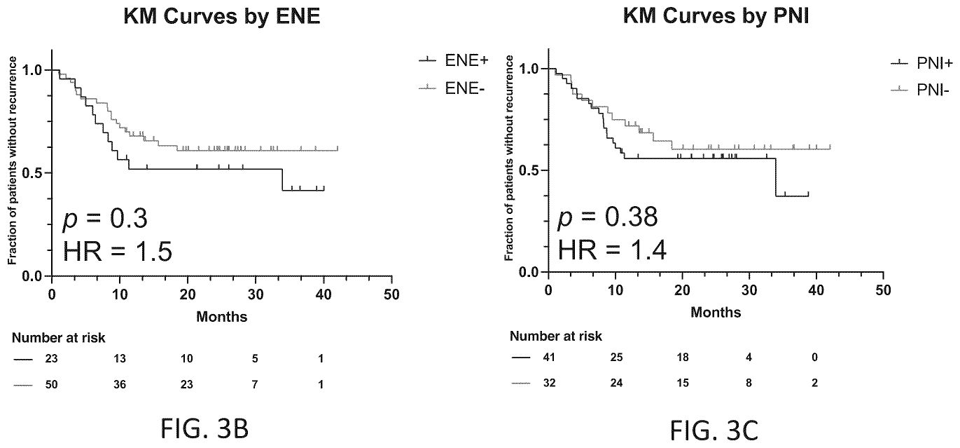

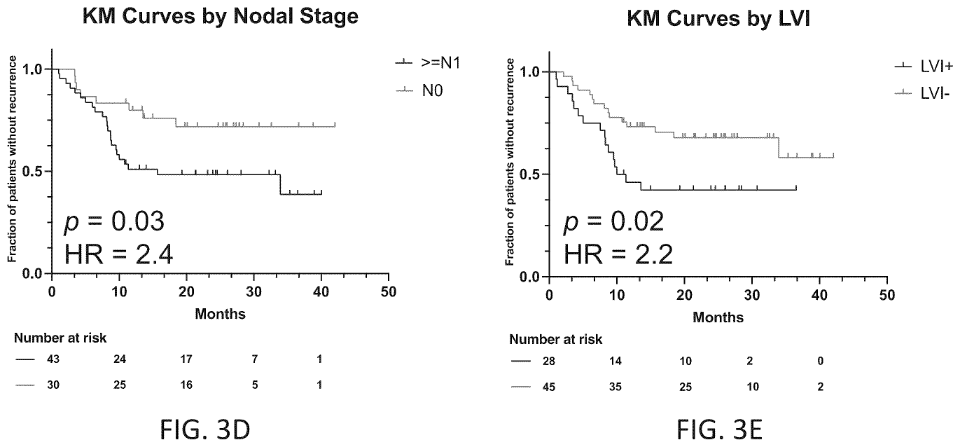

Lymph collected via surgical drains represents a novel biofluid with distinct cfDNA characteristics compared to plasma. Surgical drain fluid composition changes over time, transitioning from sanguinous to more serous (lymphatic and interstitial) fluid at 24 hours (FIG. 1B). Indeed, a distinct cfDNA characteristic was observed in postoperative lymph. cfDNA in lymph was 65 times more concentrated than plasma (mean 4.14 ng per mL of lymph, mean 0.06 ng per mL plasma, P<0.0001) (FIG. 1D). Additionally, lymph cfDNA displays a distinct nucleosome distribution and a higher dinucleosome fraction compared to plasma (). The mean ctDNA allelic fraction was 2 times higher in lymph than in plasma samples (lymph=0.004±0.036%; plasma=0.002±0.078%; p=0.03, n=42) (FIG. 1E). Notably, ctDNA was undetectable in only 19.4% of lymph samples compared to 33.3% of plasma samples in recurred patients. Taken together, lymph represents a rich novel biofluid with substantially higher ctDNA content than plasma.

Lymph but not Plasma ctDNA Correlates with HNSCC Recurrence

A tumor-informed, targeted next-generation sequencing assay was used. To maximize sensitivity and specificity while still achieving a <2 week result turnaround time, an approach was employed in which all analytes are sequenced using the same panel, but the variant calling is performed in a tumor-informed manner.

This deep coverage next-generation sequencing assay is demonstrated to routinely detect variants down to 1:100,000. All analytes were sequenced using a 699-gene HNSCC focused panel (tumor/germline coverage>250×, plasma/lymph>2500×). Tumor and whole blood libraries were sequenced to a median of 533× deduplicated coverage (range 249×-2709×). Lymph and plasma cfDNA libraries were sequenced to a median of 6,322× deduplicated coverage (range 2,544×-13,675×).

Variant calling was then performed in a tumor-informed fashion, where patient-specific somatic SNVs (median 9, range 2-95) called in tumor were force-called to detect variants at those positions in lymph or plasma. A sample was classified as positive if the mean ctDNA variant allele fraction (VAF) exceeded the calibrated MRD cut-off (see FIG. 1A).

Kaplan-Meier survival analysis was run using the MRD status above in both lymph and plasma samples from the initial study cohort. Lymph ctDNA positivity (Lymph+) accurately identified HNSCC recurrence (n=36, sensitivity=76%, specificity=30%; p0.01). The hazard ratio (HR) for disease recurrence in Lymph+ patients was 3.8, indicating that disease recurrence was more than 3 times more likely among patients with ctDNA-positive lymph samples than among those with ctDNA-negative lymph samples during the follow-up period. These results were replicated in an independent cohort of 37 patients. Again, lymph ctDNA positivity accurately identified HNSCC recurrence (sensitivity=65%, specificity=70%; p=0.04, HR=2.8), suggesting that lymph profiling is a reproducible approach to detect MRD 24 hours after surgery.

To evaluate the potential benefit of a proximal fluid on local relapse patterns, patients with locoregional relapse were compared with those with no recurrence in both the initial and replication cohorts. Detection of ctDNA in lymph was strongly associated with recurrence in both cohorts (Cohort 1: n=31, sensitivity=83%, specificity=63%; p=0.01, HR=6.0; Cohort 2: n=31, sensitivity=73%, specificity=70%; p=0.02, HR=4.5). The reproducible enhanced sensitivity for locoregional recurrence observed in these cohorts is consistent with proximal fluid being enriched for tumor-associated analytes in the setting of local tumor growth. In contrast, peripheral plasma studies have reported decreased sensitivity in the setting of locoregional recurrence.

Forty-two of the patients across these cohorts had matched plasma drawn 24 hours after surgery. Applying the same approach used for lymph, plasma was not predictive of MRD at this timepoint (sensitivity=35%, specificity=72%; p=0.7, HR=1.2). Lymph correctly identified MRD twice as often in the matched cohort (sensitivity=71%, specificity=72%; p=0.005, HR=4.0; PPV=63%, NPV=78%). As anticipated from the literature, plasma is also not predictive of locoregional disease (n=34, sensitivity=32%, specificity=72%; p=0.86, HR=1.1). The Spearman's rank correlation coefficient between VAFs in matched cohort showed a weak correlation for lymph and plasma samples (P=0.0005, r²=0.03)

Lymph ctDNA Independently Outperforms Plasma through Orthogonal Validation

To complement these findings, these results were sought to orthogonally validated through independent analysis of Cohort 1 plus two additional patients with the clinically-validated Labcorp Plasma Detect (PD) MRD assay, which represents a tumor-informed, whole-genome sequencing approach. The Spearman's rank correlation coefficient between lymph mVAF and PD ctDNA level and Fisher's exact test between MRD prediction by lymph mVAF and PD showed excellent concordance for lymph samples evaluated through both methods (mVAF vs ctDNA level P<0.0001, r²=0.49, Fisher's exact test P=0.0001).

Lymph-derived ctDNA status determined by PD again accurately identified disease recurrence (n=30, sensitivity=50%, specificity=88%; P=0.003, HR=4.8), consistent with our previous results using targeted sequencing applied to the same patient cohort (sensitivity=71%, specificity=75%; P=0.006, HR=4.5). In the sub-cohort of 14 NO stage patients, the PD assay applied to lymph cfDNA also demonstrated strong association with recurrence (sensitivity=75%, specificity=90%; P=0.004, HR=14.5).

Survival analyses were then performed using the PD MRD status derived from plasma samples for 24 patients where matched lymph samples were available. Lymph MRD status again accurately identified disease recurrence in this subgroup (n=24, sensitivity=45%, specificity=92%; P=0.003, HR=6.0) and also outperformed plasma MRD status at this timepoint (n=24, sensitivity=27%, specificity=92%, P=0.03, HR=4.2), replicating the observed reduced performance of plasma for determination of MRD status at the 24-hour post-surgery timepoint.

### Lymph MRD Prediction Outperforms Individual Pathology Features and Shows Synergy in a Combination Model

Relevant clinical features (e.g. ENE status, tumor stage, nodal stage, etc.) were compared between our two cohorts and demonstrated no significant differences between these populations. Having established their similarity, they were the pooled into a single 73 patient group to better power subgroup analyses. In aggregate, the pooled cohort demonstrated 71% sensitivity and 67% specificity in to detect HNSCC recurrence (HR=3.2, p=0.001) and again demonstrated enhanced locoregional performance (n=62, sensitivity=78%, specificity=67%, P=0.0004, HR=5.1).

Multivariate Cox regression analysis was performed with lymph ctDNA status and relevant clinical features for PFS. Only lymph ctDNA status and nodal status had significant P values, indicating minimal confounding effects for lymph ctDNA due to clinical features. A similar lack of confounding effects was observed for OS. Enhanced locoregional performance (sensitivity=79%, specificity=58%, P=0.014, HR=4.4) was again observed for OS.

The prognostic value of lymph ctDNA was evaluated in lower-risk pathologies, including patients without ENE, NO patients, and patients with T1-T2 disease. Lymph ctDNA performed consistently in the NO (P=0.11, HR=3.5) and T1-T2 (P=0.3, HR=1.9) population, similar to the full cohort (albeit not significant in this small N), suggesting lymph ctDNA detection may be a relevant biomarker in earlier stage patients. Furthermore, lymph ctDNA was strongly associated with recurrence in patients without ENE (n=50, sensitivity=80%, specificity=67%; P=0.001, HR=5.2), demonstrating that lymph ctDNA can identify patients at higher risk of recurrence despite their having lower risk pathology.

NCCN guidelines incorporate specific high-risk pathology features (e.g. ENE, perineural invasion or node status) to guide adjuvant treatment. To understand how lymph MRD compares with the performance of these features in the above patient cohort, the correlation of each with recurrence was evaluated. While two of these pathology features (positive lymph nodes and lymphovascular invasion) were statistically associated with disease recurrence in this cohort, each was less predictive of recurrence than lymph ctDNA (HR 0.95-2.31 vs. 3.08). A simple logistic regression model combining all 4 pathology features was significantly associated with recurrence (sensitivity=0.44, specificity=0.85, HR=2.8, p=0.002) but still underperformed compared to the lymph ctDNA assay.

Since lymph is collected the day after surgery, these MRD results are available on a similar timeframe to pathology results. To determine whether lymph MRD synergizes with high-risk pathology feature classification, lymph ctDNA detection was added to the 4 pathology feature logistic regression model above. When lymph ctDNA was considered alongside pathology features, recurrence prediction was further improved compared to pathology or lymph ctDNA status alone (sensitivity=0.68, specificity=0.74, HR=3.5, P=0.0003), indicating synergy between pathologic and molecular features.

Next, it was determined whether lymph ctDNA is a useful marker to identify MRD in patients with two lower-risk pathologic features (NO, ENE negative). Lymph ctDNA identifies MRD equally well in the NO population as in the full cohort (HR=3.39), suggesting lymph ctDNA detection may be a relevant biomarker in earlier stage patients. Additionally, lymph ctDNA is strongly associated with recurrence in patients without ENE (HR=4.6, sensitivity=0.79, specificity=0.65, p=0.003), suggesting that lymph MRD can identify patients at higher risk of recurrence despite their having lower-risk pathology.

To assess whether lymph MRD could have utility for therapy prediction, patients were stratified by the adjuvant therapy regimen received (RT vs. CRT). RT is typically prescribed for patients with intermediate risk while CRT is a more aggressive treatment usually reserved for patients with increased likelihood of recurrence based on pathology. Lymph MRD is strongly associated with recurrence in patients who received RT only (HR=4.81, p=0.009). In contrast, lymph+ patients who received maximal therapy (CRT) had an 80% increase in median PFS compared to lymph+ patients receiving RT alone. There was no statistical difference in PFS for lymph-patients between regimens. This indicates clinical utility for a lymph-based MRD test to identify patients who could benefit from escalated therapy.

To illustrate the potential utility of Lymph MRD testing to impact clinical care, presented below are two clinical vignettes that focus on patients with pathologically low-risk disease who underwent curative-intent tumor resection surgery. Both were pT2NO, with neither positive surgical margins or evidence of ENE. Patient DF067 received 70 Gy of adjuvant RT but no chemotherapy. One day after surgery, postoperative lymph was positive for ctDNA. Thirteen months later, DF067 suffered a locoregional recurrence in the right palate.

As an illustrative example, Patient DF165 did not receive adjuvant therapy. The 24-hour lymph sample was positive for ctDNA. Three months after surgery, the patient recurred in the left buccal mucosa. In both cases, postoperative lymph MRD tests were consistent with eventual relapse. These data illustrate that some patients with low-risk pathology will still experience relapses that pathology alone cannot predict but are detectable at the molecular level. Lymph MRD detection represents a novel and complementary facet to relapse risk prediction that in the future could be used as an adjunct to pathology to better tailor adjuvant therapy selection.

Overall results demonstrate that lymph has a 65-fold higher cfDNA yield per mL and a 2× higher tumor allelic fraction than time-matched plasma. Additionally, the average volume of surgical fluid obtained at 24 hours in HNSCC is 30 mL, meaning that it would require an impossible 3.9 liters of blood to obtain the equivalent amount of ctDNA from plasma as can be extracted from a single timepoint of routine surgical drainage. With a 130-fold advantage in available tumor-derived DNA, lymph MRD will disproportionately benefit from emerging ultra-sensitive NGS methods with <0.0001% VAF detection and beyond. The fundamental properties of lymph have the potential to overcome the sensitivity challenges faced by plasma testing in the immediate post-operative setting.

Postoperative ctDNA from surgical lymphatic fluid is a novel approach to detect MRD in patients with HPV-independent HNSCC and demonstrate that lymph outperforms plasma at this timepoint. Lymph ctDNA was significantly associated with recurrence detection, findings which then replicated in an independent multi-site cohort. Strikingly, lymph ctDNA positivity accurately predicted HNSCC recurrence with a sensitivity of 71% and a specificity of 64% (p<0.003) in the full cohort, whereas plasma was not significant at this timepoint with a sensitivity of only 33%, capturing MRD in less than half as many patients.

This prognostic effect was more pronounced for patients with locoregional relapse. Local recurrence was 5-fold more likely among patients with ctDNA-positive lymph samples than among those with ctDNA-negative lymph samples during the follow up period. Accurate MRD identification in patients with locoregional disease suggests that post-surgical lymph analysis has the potential to detect residual cancer before it spreads beyond the original tumor site and adjacent lymph nodes. This improvement in detection in the setting of local recurrence is the opposite of what has been reported in peripheral MRD assays, such as plasma, suggesting that lymph testing may enable MRD detection for the most frequent form of relapse in HNSCC.

In addition, lymph outperforms specific high-risk pathology features or a combination of them at predicting recurrence. Lymph MRD correlates with recurrence in patients with low and intermediate-risk pathology features such as ENE-negative disease with a sensitivity of 71% and a specificity of 64% (p<0.003). Thus, a molecular test according to the invention further stratifies recurrence risk. When combined, lymph and a model of four high-risk pathology synergize to outperform pathology or lymph alone. Given the 24-hour sampling, lymph MRD test results could be reported less than about 2 weeks after surgery, on a similar time scale to traditional pathology reports. This combination of synergy and synchronous timing indicates that post-surgical lymph testing enables MRD findings to be used in concert with traditional pathology measures to provide more accurate, personalized adjuvant treatment decisions.

### Sample Collection, Processing and DNA Extraction

All patients underwent Jackson Pratt (JP) surgical drain insertion during surgery. Lymph was collected from JP drains in 50 mL conical tubes approximately 24 hours after surgery. Collected fluid was transported on ice for processing. The fluid was filtered through a 40 μm cell strainer to remove clumps and debris. K2 EDTA (8 mM) was added to stabilize cfDNA, inhibit cell coagulation and inactivate nucleases. K2 EDTA-stabilized lymph was centrifuged at 2,000× g for 10 minutes to separate cfDNA-containing supernatant from cells and debris. The clarified supernatant was collected and aliquoted into 2 mL microtubes. The supernatant was stored at −80° C. For bilateral dissections involving two surgical drains, lymph was collected and processed from the side containing biopsy-proven or suspicious lymph nodes.

Peripheral blood was collected in BD Vacutainer K2 EDTA tubes (BD Biosciences, NJ) concurrently with lymph collection. Collected blood was centrifuged at 1,200× g for 10 minutes. The plasma (top) layer was transferred to a 15 mL conical tube and centrifuged for 1,800× g for 5 minutes. Clarified plasma was transferred, mixed and aliquoted into 2 mL microtubes. Plasma was stored at −80° C. Plasma-depleted blood was either mixed and aliquoted into microtubes (PDWB) or the buffy coat was pelleted (WBC). Both PDWB and WBC were stored at −80° C.

Tumor tissue was collected after surgery, snap-frozen in liquid nitrogen, embedded in optimal cutting temperature (OCT) compound blocks on dry ice and stored at −80° C.

Collected samples were processed as previously described.

The cfDNA was extracted from 1-4 mL of plasma using the QIAmp Circulating Nucleic Acid Kit (Qiagen, DE). For germline control genomic DNA extraction, 150 μL of PDWB or WBC cell pellet was extracted using the DNeasy blood and tissue kit (Qiagen, DE).

Lymph cfDNA was extracted as follows: 250 μL of lymph was centrifuged at 18,800× g for 5 minutes at 4° C. and 200 μL transferred to a new tube. The clarified lymph was treated with 3 mL proteinase k (Qiagen, DE) and 10 μL SDS 20%) (Thermo Fisher Scientific, Waltham, MA) and incubated at 60° C. for 20 minutes. cfDNA was then isolated from supernatant following incubation with 0.6× volumetric ratio of SPRISelect beads (Beckman Coulter, Brea, CA). Isolated cfDNA was purified using Zymo Select-a-size DNA Clean and Concentrator (Zymo Research, Irvine, CA) and eluted in 80 μL of Zymo elution buffer.

150 mL of whole blood or one blood cell pellet was used for genomic DNA extraction with the QiAmp DNeasy blood and tissue kit (Qiagen, DE). Tumor tissue DNA was extracted using the QIAmp DNeasy blood and tissue kit (Qiagen, DE).

DNA concentrations were quantified using the Qubit HS dsDNA Assay (Thermo Fisher Scientific, Waltham, MA) and an internal lambda DNA control was used to ensure consistent performance (quantified concentration within 20% of known concentration). DNA quality was assessed using the Agilent Tapestation Genomic DNA and cfDNA Analysis kits (Agilent Technologies, Santa Clara, CA). Following DNA extraction, the following quality control metrics were utilized for inclusion into library prep: cfDNA concentration>0.4 ng/μL; genomic DNA concentration>1 ng/μL.

### Next-Generation Sequencing

Genomic DNA was fragmented to 150 bp with the Covaris ME220. 80 ng of cfDNA or 200 ng of genomic DNA was prepared using the xGen cfDNA and FFPE Library preparation kit (Integrated DNA Technologies, Coralville, IA). 600 ng of prepared library was pooled up to 8-plex and hybridized using a standard hybridization reagent kit and a custom hybridization panel covering 699-genes of the most frequently mutated genes in HPV-independent HNSCC (Twist Bioscience, San Francisco, CA). Pre-hybridized and hybridized libraries were assessed using DS1000 DNA Screentape (Agilent Technologies, Santa Clara, CA) and quantified using Qubit HS dsDNA Assay (Thermo Fisher Scientific, Waltham, MA).

Coriell DNA samples NA12878 and NA12877 (Coriell Institute, NJ) were used as process controls as follows. NA12877 DNA was spiked into NA12878 DNA at 5% for genomic DNA, and 0.5% for cfDNA, and processed in each library prep batch. The following quality control metrics for pre-hybridized libraries were used for inclusion into hybridization: Concentration>10 ng/μL (Qubit HS dsDNA) and average fragment size>250 bp (Tapestation DS1000), process control>250 bp average fragment size (Tapestation DS1000), process control concentration>10 ng/μL (Qubit HS dsDNA). The following quality control metrics of hybridized libraries were used for inclusion into sequencing: Concentration>10 ng/μL (Qubit HS dsDNA) and average fragment size 300-500 bp (Tapestation DS1000), process control 300-500 bp average fragment size (Tapestation DS1000), process control concentration>10 ng/μL (Qubit HS dsDNA).

Hybridized libraries were sequenced on a NovaSeq 6000 (Illumina, San Diego, CA). The following sequencing run quality control metrics were used for inclusion into analysis: Q30 >85%, FASTQ yield>3 Gb (tumor and whole blood) or >180 Gb (lymph or plasma). Whole blood and tumor libraries were sequenced to 500× deduplicated coverage. Plasma and Lymph cfDNA libraries were sequenced to 5000× deduplicated coverage.

### Tumor-Informed Variant Calling

Raw reads were demultiplexed using BCL Convert (Illumina, San Diego, CA). Sample matching was performed with a custom pipeline. The pipeline was designed to confirm the identity of FASTQ files by aligning a random subset of reads and comparing highly variable sites with those in BAM files with confirmed identity using Picard (Broad Institute, Cambridge, MA). The comparison generates a LOD score. A score >=5 indicates a match, and a score<5 indicates a mismatch or contamination. Consensus read-calling and alignment of reads with UMI family size over 2 were performed as previously described. Coverage analysis was performed using Picard. Tumor and blood (PDWB or WBC) samples with median target coverage lower than 250× were excluded from further analysis. Lymph and plasma samples with median target coverage lower than 2500× were excluded from further analysis.

Tumor variant calling was performed as previously published. A panel of normals was created with 50 whole blood samples following GATK best practices. From these variants, somatic mutations on coding sequences were identified using Ensembl Variant Effect Predictor annotation. Somatic mutations with depth<50× or variant allele fraction (VAF)<5% were excluded. Somatic mutations on sex chromosomes were also excluded. Matched whole blood samples were used to filter out germline mutations or mutations driven by clonal hematopoiesis. All remaining somatic mutations identified in tumor were directly genotyped in matched lymph and plasma samples. Mutated gene frequencies were estimated in 415 HPV-independent HNSCC tumor samples from The Cancer Genome Atlas (TCGA) and compared with mutational landscape of the tumor and lymph samples in this study.

A base-specific error model (BEM) was estimated at each somatic mutation position to quantify the background noise for single nucleotide variants (SNVs). Models for lymph were built using a total of 91 high-quality lymph samples sequenced to a median coverage of 6,328× (range 2,544× to 11,580×), while models for plasma were built using a total of 49 plasma samples sequenced to median coverage of 6,191× (range 2,911× to 13,675×). BEM was fit by Weibull distribution if more than 5 non-zero non-reference alleles were observed at a tumor variant position across the reference samples. Otherwise, Gaussian distribution was used to fit the BEM. All models were fit using the SciPy package.

For each model, the BEM cutoff was defined as the 75th percentile of the distribution, which was adjusted accounting for zero-inflated reference data. The 75th percentile was used to control the false discovery rate (FDR) so that true variants would not be filtered out based on our observations through reference data. For indels, the cutoff was defined as 0.1% considering the minimal coverage required to achieve at least 2 non-reference reads. For each variant in lymph and plasma samples, VAF was set to 0 if not greater than the BEM cutoff for SNVs and indel cutoff for indels. Mean VAF was calculated for each lymph and plasma sample using all somatic mutations that passed filtering in the tumor of the patient. Somatic variants that were not detected in fluid were input into the mean VAF calculations as “0”. For the contrived process control, externally validated high confidence SNVs in NA12877 were quantified. The metric used for qualification of processing from library prep to variant calling VAF variation≤20% of expected VAF, based on NA12877 spike-in percentage.

Sample matching was performed with a custom pipeline using allele fractions of known single-nucleotide polymorphisms (SNPs) to identify samples from the same individual. Consensus reads were called with fgbio (Fulcrum Genomics, Boulder, CO) based on UMIs extracted. The reads were aligned to the hg38 human genome using the Burrows-Wheeler Aligner (BWA) as part of the nf-core/sarek pipeline. Coverage analysis was performed using Picard (Broad Institute, Cambridge, MA) on bam files generated.

Mutect2 (Broad Institute, Cambridge, MA) was used to call variants in the tumor samples with matched normals and a panel of normals. The panel of normals was created with 50 whole blood samples following GATK best practices. From these variants, somatic mutations on coding sequences were identified using Ensembl Variant Effect Predictor annotation. All somatic mutations were then used to force call mutations in matched lymph and plasma samples. Matched whole blood samples were used to filter out germline mutations or mutations driven by clonal hematopoiesis. Force-called variants were considered artifacts if the VAF was not greater than BEM cutoff controlled by false discovery rate. Mean VAF was calculated for each lymph and plasma sample counting all somatic mutations of the patient.

### Data Analysis

Python 3.8.10 was used for all subsequent analyses. Mann-Whitney U test was used for group comparisons between lymph and plasma samples, and comparisons between REC and NED patients. Due to differences in observed tumor mutation count and sequencing coverage, a unified MRD cutoff would bias the MRD prediction for patients with higher mutation rates or low coverage. Therefore, correction factors were applied to the cutoff (C) to account for tumor mutation count (Nₜ) and mean coverage depth (Dₘ) for each lymph or plasma sample. A calibrated MRD cutoff (C) was estimated for each lymph or plasma sample based on its sequencing depth and somatic mutation count in corresponding tumor sample using the formula below:

$$C = \frac{2}{{N}_{t}} \times \frac{2}{{D}_{m}}$$

where Nₜ is somatic tumor mutation count of the patient and Dₘ is average per base depth of Nₜ mutations in the patient lymph or plasma sample. cfDNA samples with mean VAF greater than the cutoff were predicted as MRD positive, and those did not were predicted as MRD negative.

To demonstrate the robustness of the calibrated MRD cutoff, one lymph sample was randomly downsampled with high observed tumor mutation count and coverage. Three technical replicates were created for downsampling to demonstrate consistency with different downsampling seeding. These data indicate that with the use of the coverage-and mutation count-corrected cutoff, consistent, accurate MRD calls were able to be made despite variation in the observed tumor mutation count and achieved sequencing coverage of the lymph sample.

A logistic regression model including 5 high-risk pathologic features (margins, ENE, PNI, LVI, and nodal status) was built as the baseline. All 5 pathologic features were binary. Another logistic regression model combining lymph ctDNA status with these 5 pathologic features was built for comparison. To avoid overfitting, Internal 5-fold cross-validation was repeated performed for both models 100 times to calculate the average prediction scores for each patient. Youden's index was calculated to serve as the MRD cutoff for prediction scores. For each model, survival analysis was performed based on average prediction scores to examine the model performance.

DNA from two Coriell cell lines (NA12877 and NA12878) was prepared with the custom hybrid capture panel, sequenced and aligned to the human reference genome, using the methodology described above for tissue and lymph samples. NA12877 was downsampled to 1/1000, 1/10000, and 1/100000 reads first, and then merged with NA12878 to create 3 samples at different mixing ratios. Three replicates were created at each downsampling ratio, resulting in a total of 9 samples. Variant calling was performed on known heterozygous and homozygous mutations of NA12877 in these 9 samples to determine the sensitivity of the assay at ultra-low VAFs.

Following binary prediction by the model, the Kaplan-Meier estimator with log-rank test and Cox proportional-hazards model were used for survival analysis. For clinical features, missing values were imputed by mode except for tumor size where median was used in covariate analysis and Cox regression analysis. Not all high-risk features had missing values. Sensitivity and specificity were calculated based on ultimate disease progression status. Mann-Whitney U test and Wilcoxon signed-rank test were used for group comparison. Student's t-test, Fisher's exact test, and Spearman's rank correlation coefficient were used to rule out confounding effects by clinical and pathological features. A p-value≤0.05 was considered statistically significant for all statistical tests.

### Orthogonal Validation

Labcorp Plasma Detect is a clinically validated, tumor-informed MRD test using ctDNA whole-genome sequencing (Baltimore, MD), and was utilized for orthogonal validation. Briefly, whole-genome sequencing was performed from tumor tissue, PDWB, plasma, and lymph-derived DNA using up to 200 ng, 50 ng, 10 ng, and 10 ng of input material, respectively, using the Illumina NovaSeq 6000 platform. Tumor-specific SNVs were identified from tumor and germline datasets, from which a candidate variant set was used to determine the presence of ctDNA within lymph or plasma DNA through a random forest machine learning model.

Specifically, candidate tumor-specific SNVs identified in the test sample were scored (ranging from 0 to 1) using a random forest machine learning algorithm trained using the caret package (v6.0.90) within the R statistical computing environment (v4.1.1). Model training occurred independently of the clinical cohort, using a combination of contrived reference cell line and noncancerous donor specimens, ultimately leveraging 500 trees, approximately 7-8 levels, and approximately 150,000 leaves.

To avoid overfitting, model training utilized 5-fold cross validation and limited the number of selected variables per split procedure (hyperparameter mtry) to the square-root of the total number of input features. Finally, variants present in properly paired mapped fragments with a random forest score>0.25 were further assessed, requiring an alternate read mapping quality>30 and a read-based mutation rate≤5.

ctDNA status was determined based on the aggregate level of signal observed across all candidate variants compared to a commercially procured reference population of noncancerous donor plasma samples [n=80, obtained under Institutional Review Board approval from Discovery Life Sciences (Alabama, USA)] and a cutoff of one standard deviation above the maximum observed value was required to report a plasma or lymph sample as ctDNA positive. An estimated tumor fraction was then calculated for each positive sample based on the aggregate variant allele observations observed as a proportion of the total unique coverage of all individual tumor-specific variants assessed.

Lymph, plasma, and blood (PDWB or WBC) were collected 24 hours postoperatively from 38 HPV-independent HNSCC patients (stages I-IV) along with resected tumor specimens. Patient-matched tumor, germline (blood), and cfDNA from lymph or plasma samples were evaluated for orthogonal validation and sequenced at approximately 150×, 30×, and 30× depth, respectively. 5 tumor and 1 lymph samples failed whole genome library preparation QC (4.6 nM and 1.4 nM, respectively); 2 lymph samples completed Plasma Detect processing, but did not complete Droplet hybrid capture processing due to insufficient DNA.

Therefore, 30 patients remained, of which, 13 patients experienced disease recurrence and 17 demonstrated no evidence of disease with >1 year of follow-up. Twenty-four of these patients with lymph samples processed by Labcorp Plasma Detect also had matched plasma samples available to enable direct comparisons. KM estimator with log-rank test and Cox proportional-hazards models were used for survival analyses.

What is claimed is:

1. A method for predicting minimal residual disease, the method comprising the steps of obtaining a lymphatic exudate in proximity to a tumor;
analyzing the lymphatic exudate to identify cfDNA that is detectable in the lymphatic exudate but that would be substantially undetectable in a blood sample obtained contemporaneous with the lymphatic exudate; and
isolating the cfDNA;
determining a sequence of the cfDNA; and
predicting minimal residual disease based on results of the determining step.

2. The method of claim 1, wherein the determining step comprises sequencing the cfDNA.

3. The method of claim 1, wherein the cfDNA is present in the lymphatic exudate in an amount between about 50-fold and 75-fold higher than an amount in the blood sample.

4. The method of claim 1, wherein the lymphatic exudate is obtained during a surgical procedure.

5. The method of claim 1, wherein the lymphatic exudate is obtained within 48 hours of a surgical procedure.

6. The method of claim 1, wherein the method produces a result with a sensitivity of at least 70% for cfDNA found in lymphatic exudate but not in blood.

7. The method of claim 1, wherein the minimal residual disease is indicative of a locoregional relapse.

8. A method for stratifying risk of recurrence in cancer patients, the method comprising the steps of:
obtaining a lymphatic exudate sample from a patient;
analyzing the sample to identify ctDNA that is detectable in the sample but not in a contemporaneously-obtained blood sample;
determining a sequence of the ctDNA; and
stratifying risk of cancer recurrence by analyzing the sequence of the cfDNA.

9. The method of claim 8, wherein the stratifying step further comprises comparing the cfDNA to a database of associates with increased risk of recurrence.

10. The method of claim 8, wherein the stratifying step is based on an amount of cf DNA identified in the sample.

11. The method of claim 1 further comprising analyzing a non-DNA based indicator of disease.

12. The method of claim 11, wherein the non-DNA indicator of disease is extranodal extension.

13. The method of claim 1, further comprising the step of determining a therapy.

14. The method of claim 1, wherein the method is repeated longitudinally.

15. The method of claim 1, further comprising an adjunct diagnostic procedure.

16. The method of claim 15, wherein the adjunct diagnostic procedure is selected from MRI, X-Ray, CT scan, tissue pathology and proteomic analysis.

17. The method of claim 1, wherein the cancer is HPV-negative head and neck squamous cell carcinoma.

18. The method of claim 1, wherein the lymphatic exudate is obtained via an implanted drain.

19. The method of claim 1, wherein the cfDNA is not found in a contemporaneously-obtained blood sample.

20. The method of claim 1. wherein the cfDNA is first detectable in blood about two weeks subsequent to being detectable in the lymphatic exudate.
